(19) United States
(12) Patent Application Publication
(10) Pub. No.: US 2021/0156868 A1
(43) Pub. Date: May 27, 2021

(54) COMPOSITIONS AND METHODS FOR DETECTING ALBUMIN

(71) Applicant: Tournament BioVenture LLC

(72) Inventor: Shaoqiu ZHUO, Moraga, CA (US)

(21) Appl. No.: 16/953,366

(22) Filed: Nov. 20, 2020

Related U.S. Application Data
(60) Provisional application No. 62/938,334, filed on Nov. 21, 2019.

(51) Int. Cl. G01N 33/68; G01N 21/31

(52) U.S. Cl. CPC G01N 33/68; G01N 21/31

(57) ABSTRACT

The present disclosure provides a method for determining the amount of albumin in a sample. In one embodiment, the method involves treating the sample with an esterase inhibitor that selectively inhibits non-albumin esterase activity; combining the sample with a selective substrate of albumin, which has a carboxylic ester bond, so that the carboxylic ester bond is cleaved to generate a hydrolysate; detecting the amount of the hydrolysate generated in a period of time; and determining the amount of the albumin in the sample based on the amount of the hydrolysate in the period of time.

### CROSS-REFERENCE TO RELATED APPLICATIONS

This application claims priority to U.S. provisional patent application 62/938,334, filed Nov. 21, 2019, the disclosure of which is incorporated herein by reference.

### FIELD OF THE INVENTION

The present disclosure generally relates to medical diagnostics. More particularly, the present disclosure relates to compositions and methods for specific detection and quantization of albumin.

### BACKGROUND OF THE INVENTION

Albumin is a family of non-glycosylated globular proteins that are commonly found in blood plasma. Serum albumin is the main protein of human blood plasma and performs a variety of physiological functions, such as maintaining the colloidal osmotic pressure, transporting various biomolecules, and exerting antioxidant action.

Albumin estimation is commonly performed in clinical biochemistry laboratories. For example, urinary albumin has been widely used as an important biomarker for patients with renal damage, such as diabetes, hypertension and poststreptococcal acute glomerulonephritis. While the normal albumin range in adult human serum is 34-54 g/L, low albumin may be associated with liver disease, nephrotic syndrome, burns, protein-losing enteropathy, malabsorption, malnutrition, late pregnancy, artefact, genetic variations and malignancy.

Many methods have been developed to quantitatively measure the albumin level, including electrophoresis, HPLC, immunochemical assay and dye binding assay. Electrophoresis method is slow, expensive, requiring relatively large sample volume and easy to overestimate the serum albumin concentration. HPLC method is unable to quantitate albumin derived fragments that are smaller than 10 KDa; other urinary proteins such as transferrin are often co-elute with albumin in size-exclusion HPLC. While immunochemical assays are specific for albumin estimation and many variations are available, the methods are generally high-cost and requiring long incubation time and washing steps. At present, some dye binding assays, such as those based on methyl orange and bromcresal green, are available. But their specificity is relatively low because other proteins in the sample also have the ability to bind these dyes.

Albumin binds and transports many hydrophobic compounds including lipids in circulation. While binding lipids or esters, certain tyrosine residues (e.g., Tyr150 and Tyr 411) of albumin can display esterase activity (called pseudo esterase). Many compounds, such as α- and β-naphthyl acetate, p-nitrophenylacetate (NPA), fatty acid esters, aspirin, keoprofen glucuronide, cyclophosphamide, esters of nicotinic acid, octanoylghrelin, nitroacetanilide, nitrotrifluoracetanilide, and organophosphorus compounds have shown to be the substrates of albumin (see Goncharov N V et al., Molecules (2017) 22:1201). Some probes, which are the substrate of albumin, have been used to assess the concentration of albumin (see, e.g., U.S. Pat. No. 9,340,821 to Yang et al). However, the specificity of the probes limits their clinical application for quantifying albumin in biological samples. Therefore, there is a continuing need to develop new compositions and methods to accurately and efficiently measure the albumin level in biological samples.

### SUMMARY OF THE INVENTION

The present disclosure in one aspect provides a method for determining the amount of albumin in a sample. In one embodiment, the method involves treating the sample with an esterase inhibitor or inactivator that selectively inhibits or inactivates non-albumin esterase activity; combining the sample with a selective substrate of albumin, which has a carboxylic ester bond, so that the carboxylic ester bond is cleaved to generate a hydrolysate; detecting the amount of the hydrolysate generated in a period of time; and determining the amount of the albumin in the sample based on the amount of the hydrolysate generated in the period of time.

In certain embodiments, the albumin is human serum albumin (HSA).

In certain embodiments, the sample is blood, plasma, serum or urine.

In certain embodiments, the selective substrate of HSA is a nitrophenyl ester of fatty acid or lipid. In certain embodiments, the selective substrate of HSA is a (p-, m-, o- or di-) nitrophenyl ester of fatty acid or lipid. In certain embodiments, the selective substrate of HSA is a p-nitrophenyl ester of fatty acid. In certain embodiments, the selective substrate of HSA is 1-myristoyl-2-(4-nitrophenylsuccinyl)-sn-glycero-3-phosphocholine (14:0 NPS PC).

In certain embodiments, the hydrolysate has a colorimetric or fluorescent property which enables effective detection of the hydrolysate via a photometric or fluorescent detector. In certain embodiments, the hydrolysate is nitrophenol. In certain embodiments, the hydrolysate is detected via absorption at wavelength of about 405 nm. In certain embodiments, the HSA pseudo esterase activity is assessed by the rate of increase at 405 nm. In certain embodiments, the HSA pseudo esterase activity is assessed by the final increase of absorption at 405 nm.

In certain embodiments, the esterase inhibitor is a compound containing fluorosulfonyl (sulfonyl fluoride) functional group. In certain embodiments, the esterase inhibitor is a compound containing a benzenesulfony fluoride functional group. In certain embodiments, the esterase inhibitor is 4-(2-aminoethyl)benzenesulfonyl fluoride hydrochloride (Pefabloc SC) or benzylsulfonyl fluoride. In certain embodiments, the esterase inhibitor is phenylmethylsulfonyl fluoride (PMSF).

In certain embodiments, the biological samples can be pretreated with the esterase inhibitor before the assay. In certain embodiments, the esterase inhibitor and the selective substrate of HSA are contained in one solution.

In another aspect, the present disclosure provides a kit for detecting determining the amount of albumin in a sample. In certain embodiments, the kit comprises an esterase inhibitor that selectively inhibits non-albumin esterase activity. The kit further comprises a selective substrate of albumin, which has a carboxylic ester bond, so that when the substrate is exposed to albumin in the sample, the carboxylic ester bond is cleaved to generate a hydrolysate. In certain embodiments, the kit further comprises a standard control of albumin.

### BRIEF DESCRIPTION OF THE DRAWINGS

FIG. 1 shows the albumin and non-albumin esterase activity in human serum. Human serum was fractionated by Superose-6 column. And the esterase activity in each fraction was identified using 14:0 NPS-PC with or without inhibitor or suppressor of HSA pseudo esterase activity. In the absence of inhibitor of HSA, three main fractions of esterase activity were identified. When the esterase activity of HSA was inhibited, the third peak disappeared, indicating that the last peak was the esterase activity of HSA.

FIG. 2 shows the result of an assay according to an embodiment of the disclosed invention.

FIG. 3 shows the Michaelis-Menten kinetics of HSA pseudo esterase using 14:0 NPS-PC as probe. 14:0 NPS-PC has relative low Km in the pseudo esterase reaction of HSA and thus the measurement of HSA concentration can be sensitive and fast.

FIG. 4 shows that as compared to the pseudo esterase activity of albumin, the esterase activity associated with LDL or HDL is much more sensitive to Pefabloc SC. Pefabloc SC at 15-20 mM (final concentration) can eliminate all non-albumin esterase activity but maintain >90% of albumin pseudo esterase activity at 1.5 mM 14:0 NPS-PC in pH 7.4.

FIG. 5 shows the different sensitivity to the incubation with 2 mM Pefabloc SC between HSA and esterase activity associated with purified LDL and HDL. The esterase activity associated with LDL and HDL were completely inactivated when incubated with 2 mM Pefabloc SC around 6-10 hr. No inactivation of HSA pseudo esterase activity was observed under the same conditions.

FIG. 6 shows the complete inactivation of human serum non-HSA esterase activity by Pefabloc SC at about 1 mM when incubated at 24° C. for 1 hr.

FIG. 7 shows that the human serum non-HSA esterase activity can be completely inactivated by incubation with 1 mM Pefabloc SC at 24° C. for about 15 min.

FIG. 8 demonstrates that the increase of HSA concentration with fixed substrate concentration results in the decrease of R² of the linearity.

FIG. 9 shows the optimal substrate/HSA ratio to obtain the best linearity of HSA quantization curve. Based on the figure, the optimal concentration of substrate can be determined for different formats of the HSA quantization assay.

FIG. 10 shows that no difference is observed for reading the assay by either the reaction rate or the end point in the quantization of HSA under the conditions.

FIG. 11 shows that albumin pseudo esterase activity can be affected by Ca²⁺ and Cu²⁺.

FIG. 12 shows the inhibition of Lp-PLA2 in human serum by darapladib.

FIG. 13 shows the effects of DMSO, EDTA and Tween-20 on pseudo-lipase activity of albumin.

FIG. 14 shows the correlation of albumin signal to assay volume of human serum.

FIG. 15 shows the standard curve and parameters.

FIG. 16 shows the bivariate fit of detected albumin (mg/mL) by expected albumin (mg/mL).

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

As used herein, the term “about” means plus or minus 10% of the numerical value of the number with which it is being used. Therefore, about 5 mg/kg body weight means in the range of 4.9 to 5.1 mg/kg body weight.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps. Terms such as “consisting essentially of” and “consists essentially of” have the meaning attributed in United States Patent law; they allow for the inclusion of additional ingredients or steps that do not materially affect the basic and novel characteristics of the claimed invention. The terms “consists of” and “consisting of” have the meaning ascribed to them in United States Patent law; namely that these terms are close ended.

A “sample” or “biological sample” refers to any sample that is taken from a subject (e.g., a human, or a subject suspected of having a condition or disease that causes abnormal level of album in serum or urine) and contains albumin, albumin homologs or orthologs that have esterase activity. The biological sample can be a bodily fluid, such as blood, plasma, serum, urine, vaginal fluid, uterine or vaginal flushing fluids, plural fluid, ascitic fluid, cerebrospinal fluid, saliva, sweat, tears, sputum, bronchioalveolar lavage fluid, etc.

A “subject” as used herein refers to warm blooded animals including human and non-human animals. Non-human animals include all vertebrates capable of naturally producing albumin esterase activity (e.g. albumin, albumin homologs or orthologs), for example, mammals and non-mammals, such as, for example, guinea pigs, mice, rats, gerbils, cats, rabbits, dogs, cattle, swine, sheep, horse and non-human primate. Preferably, the subject of the present disclosure is human. The subject may be male or female, may be elderly, and may be an adult, adolescent, child, or infant. A human subject may be Caucasian, African, Asian, Semitic, or other racial backgrounds, or a mixture of such racial backgrounds.

### Methods for Detecting Albumin

The present disclosure in one aspect provides a method for determining the amount of albumin in a biological sample based on albumin's pseudo esterase activity. “Albumin esterase activity” or “albumin pseudo esterase activity” as used herein includes, but is not limited to any esterase activity of albumin. This activity may include but is not limited to an enzyme binding substrate, releasing product, and/or hydrolyzing carboxylate esters, phospholipids or other molecules. Alternatively, albumin esterase activity can be measured against a standard recombinantly expressed, semi-purified or purified protein.

The method described herein uses two ways to increase the specificity of albumin: (1) by using a substrate or probe that specifically binds to albumin; and (2) by inactivating non-albumin esterase/hydrolase activity in the biological sample. The method is easy to apply and be adapted to various clinical instruments. The method takes short period of time of about 3-5 minutes and reduces cost significantly.

In certain embodiments, the method involves treating the sample with an esterase inhibitor that selectively inhibits non-albumin esterase activity, i.e., the esterase inhibitor preferentially suppresses the esterase/hydrolase activity of the non-albumin esterase in the sample, e.g., the esterase inhibitor suppresses at least 80%, 85%, 90%, 95%, 99% of the non-albumin esterase in the sample but suppresses less than 10%, 9%, 8%, 7%, 6%, 5%, 4%, 3%, 2%, 1% of the pseudo esterase activity of albumin. In certain embodiments, the esterase inhibitor that selectively inhibits non-albumin esterase activity reduces or eliminates any of the activities of non-albumin esterase, including, but not limited to, enzyme binding substrate, releasing product, and/or hydrolyzing carboxylate esters, phospholipids or other molecules.

In certain embodiments, the esterase inhibitor are compounds containing sulfonyl fluoride group, such as PMSF or Pefabloc SC. Examples of the esterase inhibitors that can be used in the method described herein are shown in Table 1.

In certain embodiments, the method described herein involves combining the sample with a selective substrate of albumin, which has a carboxylic ester bond, so that the carboxylic ester bond is cleaved to generate a hydrolysate. As used herein, a “selective substrate of albumin” means that the substrate is preferentially hydrolyzed by albumin in the sample, e.g., less than 10%, 9%, 8%, 7%, 6%, 5%, 4%, 3%, 2%, 1% of the substrate hydrolyzed in the sample is catalyzed by non-albumin esterase in the sample.

In certain embodiments, the selective substrate contains a colorimetric or fluorometric detectable moiety. “Colorimetric or fluorometric detectable moiety” as used herein is a portion of a compound capable of producing a detectable or measurable signal. Such a signal may be measurable by, but not limited to, visible light emission or absorption, fluorescence, phosphorescence or other detectable quanta. For instance, a substrate for albumin esterase may comprise a colorimetric moiety bonded to carboxylate esters or phosphatidylcholine at the albumin esterase cleavage site. When albumin cleaves the colorimetric moiety from the carboxylate esters or phosphatidylcholine, the colorimetric moiety emits a detectable signal as visible light or fluorescence. One non-limiting example of phosphatidyl choline bonded to a colorimetric moiety is 1-myristoryl-2-(4-nitrophenylsuccinyl) phosphatidylcholine.

In certain embodiments, the selective substrate is a nitrophenyl ester of fatty acid or lipid. Albumin is a known hydrolyzer of certain carboxylate (fatty acid) esters and phospholipids. Albumin can cleave phospholipids at the sn-2 position to create lyso-PC and fatty acids. A substrate possessing a colorimetric or fluorometric moiety can be used to measure albumin esterase activity. For instance, the substrate, 1-myristoyle-2-(p-nitrophenylsuccinyl)-phosphatidylcholine, is a carboxylic glycerol ester with a 4-nitrophenyl group conjugated onto a succinyl chain at sn-2 position. Albumin hydrolyzes the sn-2 position of the substrate, producing 4-nitrophenyl succinate. This liberation can be spectrophotometrically monitored at 405 nm and albumin esterase activity determined from the change in absorption. Using 1-myristoyle-2-(p-nitrophenylsuccinyl)-phosphatidylcholine or other lipid analogues as the substrate for albumin can reduce the binding specificity for other non-albumin enzymes and thus increase the reaction specificity for albumin. Using 1-myristoyle-2-(p-nitrophenylsuccinyl)-phosphatidylcholine or other lipid analogues as the substrate for albumin can also competitively exclude the binding of other hydrophobic compounds such as fatty acids or lipids which often present in biological samples and reduce the esterase activity of albumin and thus increase the accuracy of the assay.

In certain embodiments, the selective substrate is selected from the group consisting of: p-nitrophenyl, o-nitrophenyl or m-nitrophenyl esters of fatty acids or lipids. As used herein, the fatty acids that forms a nitrophenyl ester include, without limitation, caprylic acid, capric acid, lauric acid, myristic acid, palmitic acid, stearic acid, arachidic acid, behenic acid, lignoceric acid, cerotic acid, myristoleic acid, palmitoleic acid, oleic acid, vaccenic acid, linoleic acid, linoelaidic acid, arachidonic acid, eicosatetraenoic acid, erucic acid, and docosahexaenoic acid.

In certain embodiments, the selective substrate of HSA is 1-myristoyl-2-(4-nitrophenylsuccinyl)-sn-glycero-3-phosphocholine (14:0 NPS PC). There are at least two advantages using 14:0 NPS PC as the selective substrate: (1) high specificity for albumin because of its phosphocholine (PC) structure which limits its other specificity only to phospholipase A2 family; (2) it is highly hydrophobic and can displace the bound fatty acid esters or lipids on albumin and thus reduce the underestimation of the protein.

In certain embodiments, the method described herein involves detecting the amount of the hydrolysate generated in a period of time; and determining the amount of the albumin in the sample based on the amount of the hydrolysate in the period of time.

In certain embodiments, the period of time is 1 second, 10 seconds, 15 seconds, 30 seconds, 1 minute, 2 minutes, 3 minutes, 4 minutes, 5 minutes, 10 minutes, 15 minutes, 20 minutes, 25 minutes, 30 minutes, etc.

In certain embodiments, the hydrolysate has a chromophore property which enables effective detection of the hydrolysate via a common light detector and avoid the use of expensive fluorescence detector. In certain embodiments, the hydrolysate is nitrophenol. In certain embodiments, the hydrolysate is detected via a detector at wavelength of about 405 nm. In certain embodiments, the albumin pseudo esterase activity is assessed by the rate of increase at 405 nm. In certain embodiments, the albumin pseudo esterase activity is assessed by the final increase of absorption at 405 nm.

TABLE 1
Examples of Selective Esterase Inhibitor

| Chemical | Formal Name |
|---|---|
| 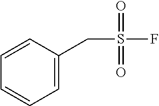 [SMILES: O=S(=O)(F)Cc1ccccc1] | Phenylmethylsulfonyl fluoride (PMSF), Phenylmethanesulfony fluoride, benzylsulfony fluoride, α-Toluenesulfonyl fluoride |
| 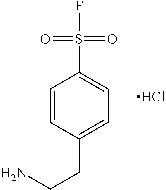 [SMILES: NCCc1ccc(S(=O)(=O)F)cc1 ; Cl] | 4-(2-Aminoethyl)benzenesulfonyl fluoride hydrochloride, Pefable SC |
| 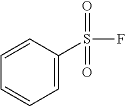 | Benzenesulfonyl fluoride |
| 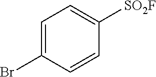 [SMILES: O=S(=O)(F)c1ccc(Br)cc1] | 4-Bromo-benzenesulfonyl fluoride |
| 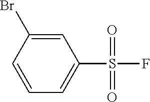 [SMILES: O=S(=O)(F)c1cccc(Br)c1] | 3-Bromobenzenesulfonyl fluoride |
| 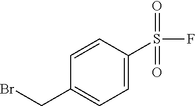 [SMILES: O=S(=O)(F)c1ccc(CBr)cc1] | 4-(Bromomethyl)benzenesulfonyl fluoride |
| 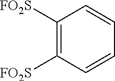 | 2-Benzenedisulfonyl fluoride |
| 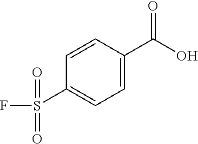 [SMILES: O=C(O)c1ccc(S(=O)(=O)F)cc1] | 4-(Fluorosulfonyl)benzoic acid |
| 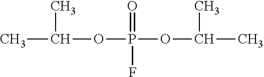 | Diisopropylfluorophosphate |
| 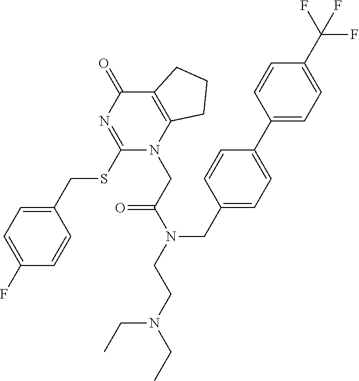 [SMILES: CCN(CC)CCN(Cc1ccc(-c2ccc(C(F)(F)F)cc2)cc1)C(=O)Cn1c(SCc2ccc(F)cc2)nc(=O)c2c1CCC2] | Darapladib N-[2-(diethylamino)ethyl]-2-[2-[(4- fluorophenyl)methylsulfanyl]-4-oxo-6,7- dihydro-5H-cyclopenta[d]pyrimidin-1-yl]- N-[4-[4-(trifluoromethyl)phenyl]phenyl] methyl]acetamide |

### Kits for Detecting Albumin

In another aspect, the present disclosure provides a kit for use in the methods described here. The kit may include any or all of the reagents to perform the methods described herein. In such applications, the kit may include any or all of the following: an esterase inhibitor that selectively inhibits non-albumin esterase activity; a selective substrate of albumin, which has a carboxylic ester bond, so that when the substrate is exposed to albumin in the sample, the carboxylic ester bond is cleaved to generate a hydrolysate; a standard control of albumin; and buffers. In certain embodiments, the kit also includes a metal ion chelator, e.g., EDTA or EGTA. In certain embodiments, the kit also includes one or more types of elements or components such as other types of biochemical reagents, containers, packages such as packaging intended for commercial sale, etc.

In addition, the kit may include instructional materials containing directions (i.e., protocols) for the practice of the methods provided herein. While the instructional materials typically comprise written or printed materials, they are not limited to such. Any materials capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The following examples are provided to better illustrate the claimed invention and are not to be interpreted as limiting the scope of the invention. All specific components, materials, and methods described below, in whole or in part, fall within the scope of the present invention. The specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that may variations can be made in the procedure herein described while still remaining within the bounds of the present invention. It is the intention of the inventors that such variations are included within the scope of the invention.

### Example 1

This example shows the albumin and non-albumin esterase activity in human serum.

Human serum was fractionated using superose-6 column, followed by the identification of esterase activity using 14:0 NPS-PC as probe with or without inhibitor of HSA. As shown in FIG. 1, in the absence of inhibitor of HSA, three main fractions of esterase activity were identified. When the esterase activity of HSA was inhibited, the third peak disappeared, indicating that the last peak was the esterase activity of HSA. The first and the second peaks were non-albumin esterase or lipase activities.

There are two advantages using 14:0 NPS-PC as probe: First, less non-albumin esterase/lipase/hydrolase activity because the unique phosphocholine (PC) structure which limits its specificity only to phospholipase A2 (PLA2). Second, it is highly hydrophobic and can displace the bound fatty acid esters or lipids on albumin and thus reduce the underestimation of the protein.

### Example 2

This example shows a method of determining the amount of albumin in a biological sample.

The reaction was started by the addition of 110 μl of TBS reaction buffer (10 mM Tris-HCl, 150 mM NaCl, pH 7.4) containing 0.5 M 14:0 NPS-PC and 5 mM EDTA to 20 μl of HSA solution in a well of a 96-well plate. The reactions were followed at wavelength 405 nm (absorbance) in a SPECTRAmax M5 plate reader.

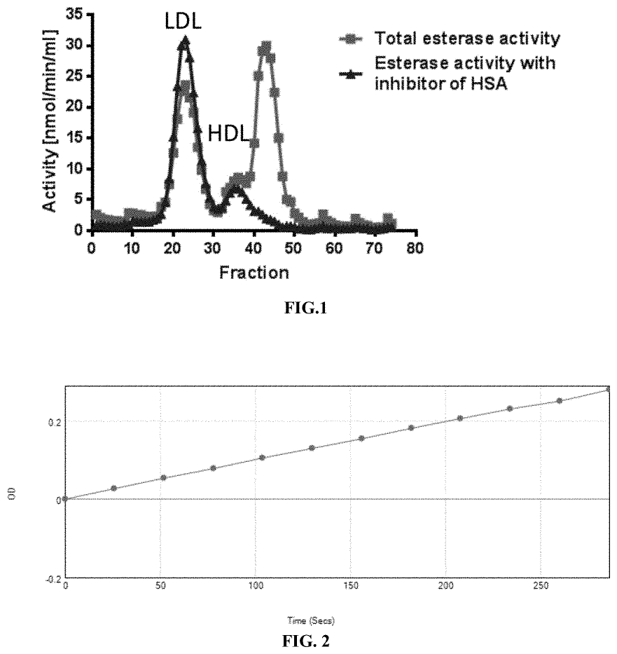

As shown in FIG. 2, the absorption at wavelength 405 nm increased while 14:0 NPS-PC was hydrolyzed by HSA to release p-nitrophenol. The method does not require pre-incubation. Reading time can be from 0.5 to 30 min depending on the concentration of albumin. Typical reading time is 3-5 min. Reaction time temperature can be ambient.

### Example 3

This example shows that 14:0 NPS-PC is an effective substrate to measure the pseudo esterase activity of albumin.

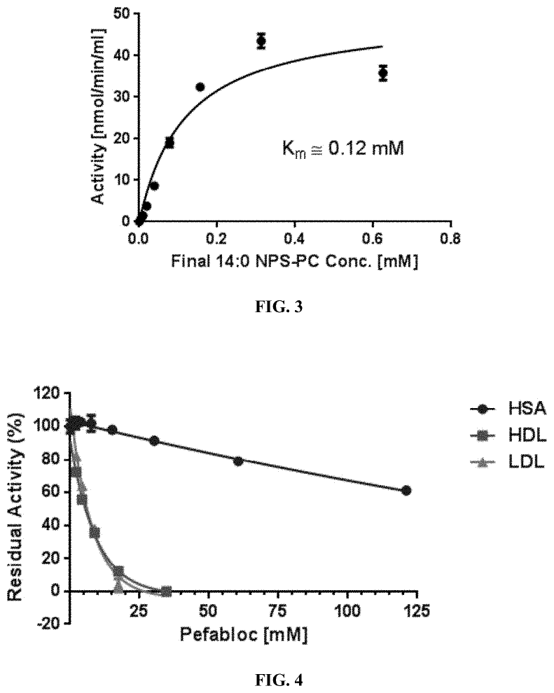

FIG. 3 shows the Michaelis-Menten kinetics of HSA pseudo esterase using 14:0 NPS-PC as probe. As shown in FIG. 3, 14:0 NPS-PC has relative low Km in the pseudo esterase reaction of HSA and thus the measurement of HSA concentration can be sensitive and fast.

### Example 4

This example shows that Pefabloc SC selectively inhibits non-albumin esterase activity in human serum.

As shown in EXAMPLE 1, using 14:0 NPS-PC as probe also identified non-albumin esterase activity, i.e. PLA2 activity in human serum, which is mainly associated with HDL and LDL. As shown in FIG. 4, comparing to the pseudo esterase activity of albumin, the PLA2 activity in human serum is much more sensitive to Pefabloc SC or PMSF or other benzenesulfonyl fluoride inhibitors or inactivators. Pefabloc at 15-20 mM (final concentration) can eliminate all non-albumin esterase activity but maintain >90% of albumin esterase activity when assayed with 1.5 mM 14:0 NPS-PC at pH 7.4. Thus, non-albumin esterase activity can be eliminated by either treating the biological samples with Pefabloc SC, or PMSF or analogue inhibitors or inactivators or by including the compound in the assay buffer.

Including 5 mM EDTA in the assay buffer also suppresses the activity of Ca²⁺ dependent PLA2 towards 14:0 NPS-PC.

### Example 5

This example also demonstrates the difference of sensitivity towards Pefabloc SC between HSA and non-HSA esterase activities in serum.

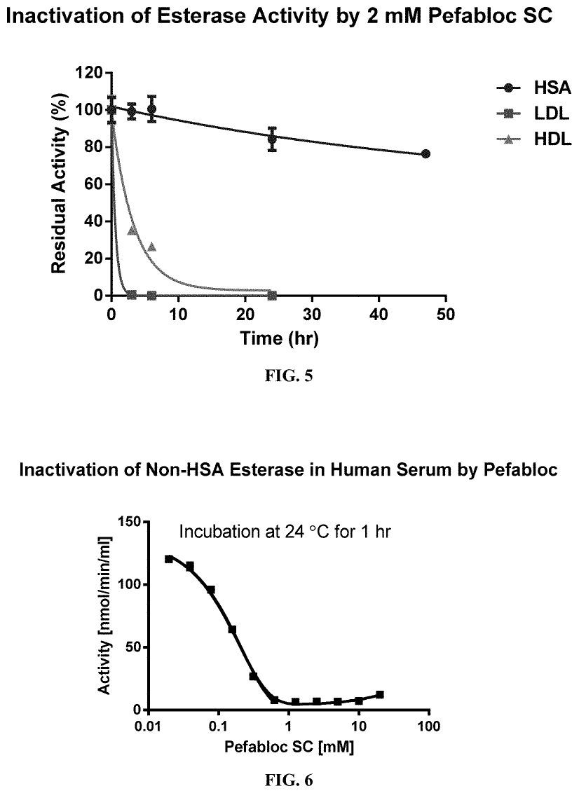

FIG. 5 shows that when treated with 2 mM Pefabloc SC, non-HSA esterase activity associated with LDL (5 mg/ml of cholesterol) was completely inactivated within 3 hr of incubation. About 80% of non-HSA esterase activity associated with HDL (2.5 mg/ml of cholesterol) was depleted in 6 hr of incubation with 2 mM Pefabloc SC but no activity loss was observed for HSA at 44 mg/ml in TBS, pH 7.4, under the same conditions.

FIG. 6 shows the sensitivity of non-HSA esterase activity in the mix of 20 human sera from healthy donors towards Pefabloc SC. The activity was completely suppressed when the serum mix was incubated with >1 mM of Pefabloc SC for about 1 hr at 24° C.

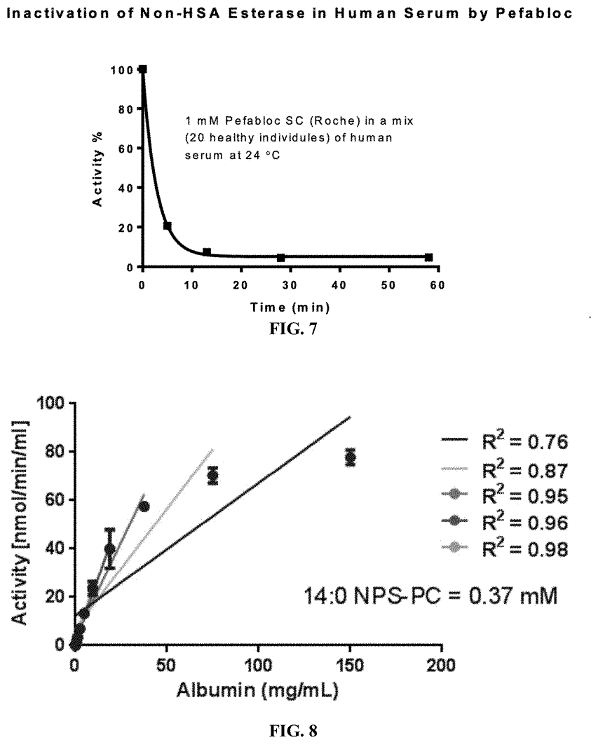

FIG. 7 also shows the sensitivity of non-HSA esterase activity in the mix of 20 human sera from healthy donors towards Pefabloc SC. When human serum was incubated with 1 mM Pefabloc, the non-HSA esterase activity can be abolished in about 10 minutes. Both FIG. 6 and FIG. 7 indicate that the non-HSA esterase activity in human sera is more sensitive towards Pefabloc SC than that in the isolated LDL and HDL. This could be due to the TBS buffer in the isolated LDL and HDL, which may reduce the effective concentration of Pefabloc SC.

### Example 6

This example shows the effect of substrate/HSA ratio in a method of the present disclosure.

The esterase activity of albumin of different concentrations was measured with 0.37 mM 14:0 NPS-PC in the assay system. As shown in FIG. 8, the linear relationship between the albumin concentration and the esterase activity decreased when the concentration of albumin increased, indicating that the linearity of the assay may depend on the ratio of substrate/albumin.

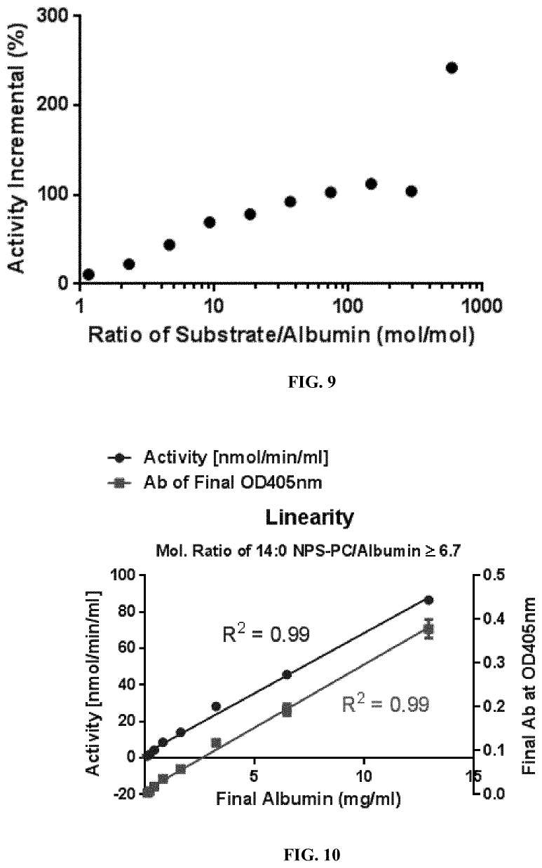

As shown in FIG. 9, in order to keep linearity of the assay, the 14:0 NPS-PC/albumin ratio should be maintained at least around 5 fold for the highest concentration of albumin to be determined. The concentration of 14:0 NPS-PC of the assay is depending on the quantity of biological samples and volume of the assay format. However, the ratio of substrate/albumin should keep constant or similar.

As shown in FIG. 10, human serum albumin can be quantified based on the incremental rate (activity) or final concentration of nitrophenol (final absorption at 405 nm). Both give excellent correlation to albumin concentration. Limit of detection (LOD) was estimated to be around 0.1 mg/ml (assay conc.) of albumin and Limit of quantization (LOQ) was estimated to be around 0.4-0.8 mg/ml (assay conc.) of albumin under the conditions. This means biological samples can be diluted between 10-100 folds for measurement of albumin concentration. Some types of bio fluid such as urine may be analyzed without dilution by this method. Bio fluids mean serum, plasma, whole blood or urea, etc. Samples can be fresh or dry.

### Example 7

This example shows that albumin pseudo esterase activity can be affected by metal ions. Albumin is known to bind certain metal ions, especially transient metal ions, such as Ca²⁺ and Cu²⁺, which commonly present in biological samples and affect the pseudo esterase activity of albumin. As shown in FIG. 11, the pseudo esterase activity of HSA can be affected by the presence of Ca²⁺ and Cu²⁺. Therefore, EDTA or EGTA or other metal ion chelator is included in the assay components to keep the consistency for all biological samples,

### Example 8

This example illustrates a method for determining albumin esterase activity in a biological sample obtained from an animal. The method comprises the steps of:

(a) Mix an assay solution with the biological sample or HAS standards. The assay solution comprises: 1-myristoyl-2-(4-nitrophenyl succinyl) phosphatidylcholine, 200 mM HEPES (alternatively, 200 mM Tris-HCl), 150 mM NaCl, 5 mM EDTA, pH 7.4-7.6. The final concentration of 1-myristoryl-2-(4-nitrophenylsuccinyl) phosphatidylcholine is dependent on the estimated concentration of albumin in the biological sample, generally, 5-10 fold (mol/mol) of the final concentration of albumin. Change of absorption at 405 nm is monitored.

(b) Calibration of esterase activity based on the following two curves:

Curve 1 is prepared by using each of a p-nitrophenol standard solution comprising 200, 100, 75, 50, 25, 10 and 5 nmol/ul p-nitrophenol in methanol; and same volume of phosphate buffered saline (PBS) or ddH₂O to make blanks;

Curve 2 is prepared by using each of a p-nitrophenol standard solution comprising 4, 3, 2, 1, 0.5, and 0.25 nmol/ul p-nitrophenol in methanol; and same volume of phosphate buffered saline (PBS) or ddH₂O to make blanks.

Step 1: generating a standard curve by plotting optical density (OD) values at 405 nm for the p-nitrophenol standard solutions vs. p-nitrophenol concentrations (nmol/well);

Step 2: calculating the slope (OD/nmol) of the standard curve;

Step 3: calculating the absorbance change between 3 and 1 minute (ΔOD3 min-1 min or longer depending on the slope change rate) for both solutions comprising biological samples and blank;

Step 4: calculating esterase activity using the following formula:

Esterase activity (nmol/min/ml)=(ΔODsample−ΔOD blank)/slope (OD/nmol)/vol (ml)/2 (minutes)

### Example 9

This example illustrates the development of an assay for detection and quantization of human albumin in serum, plasma, whole blood or urine. The assay is intended to quantify normal and disease modified human albumin in human body fluids. The specificity of the assay is based on the specific substrate structure and the suppression of lipoprotein associated phospholipase A2 (Lp-PLA2) by its inhibitors or inactivators. The components of the assay reagents have been optimized for assay of albumin in serum, plasma and whole blood samples.

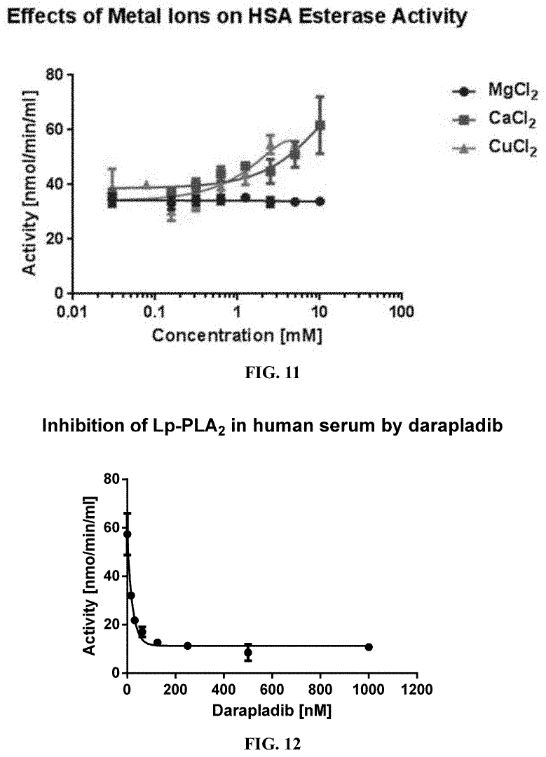

Lp-PLA2 activity is the major interference for the assay of albumin. Suppression of Lp-PLA2 activity by specific inhibitors is critical for assay of albumin. As shown in FIG. 12, darapladib suppresses Lp-PLA2 in a concentration dependent manner.

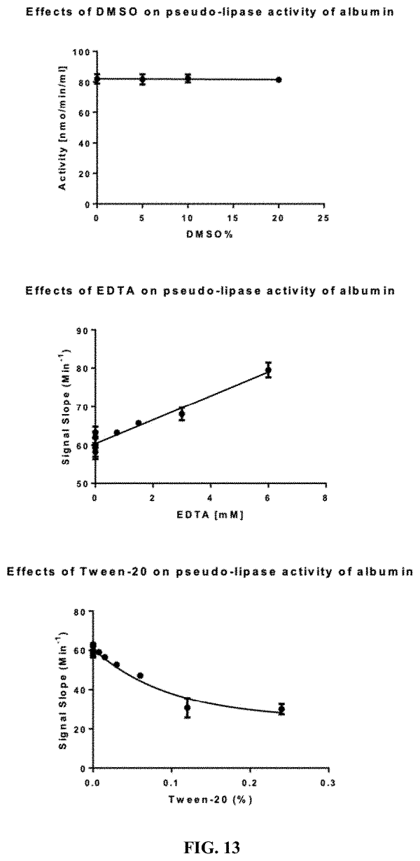

Darapladib is insoluble in water and need to be dissolved in organic solvents. Dimethyl sulfoxide (DMSO), EDTA and Tween-20 had been used to determine the tolerable concentration of organic solvent. The results showed that DMSO does not have effects on the assay of albumin (FIG. 13).

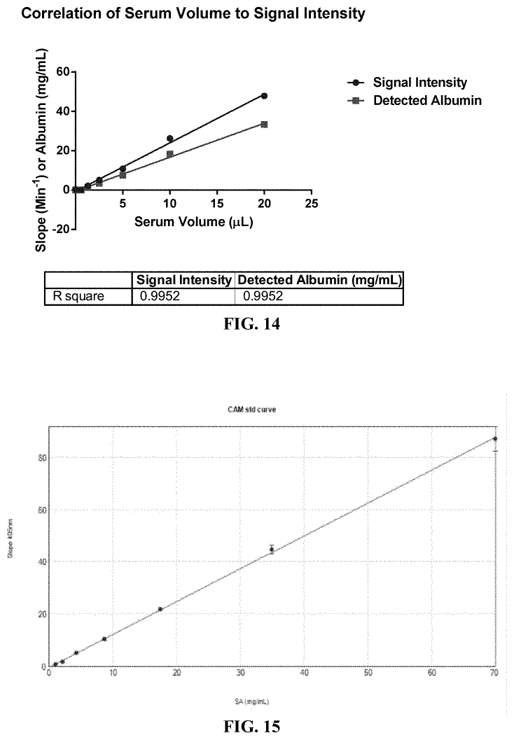

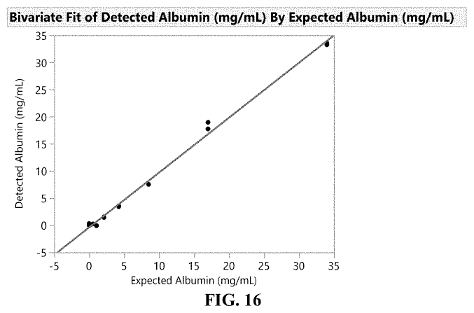

As shown in FIG. 14, albumin quantity is proportional to the signal increase in a linear correlation. The assay is also assessed for its sensitivity and limit in detection and quantization, the results of which are shown in FIGS. 15-16 and Table below.

Linear Fit
Detection Albumin (mg/mL) = −0.433927 +
1.0111833*Expected Albumin (mg/mL)

Summary of Fit

| | |
|---|---|
| RSquare | 0.995254 |
| RSquare Adj | 0.994859 |
| Root mean Square Error | 0.857803 |
| Mean of Response | 9.197143 |
| Observation (of Sum Wgts) | 14 |

Analysis of Variance

| | | Sum of | Mean | |
|---|---|---|---|---|
| Source | DF | Square | Square | F Ratio |
| Model | 1 | 1851.7608 | 1851.76 | 2516.573 |
| Error | 12 | 8.8299 | 0.74 | Prob > F |
| C. Total | 13 | 1860.5907 | | <0.0001 |

Parameter Estimate

| | | Std | t | Prob > |
|---|---|---|---|---|
| Term | Estimate | Error | Ratio | \|t\| |
| Intercept | −0.433927 | 0.299028 | −1.45 | 0.1724 |
| Expected Albumin (mg/mL) | 1.0111833 | 0.020157 | 50.17 | <0.0001 |

In one exemplary embodiment, the formulation of the albumin detection and quantization assay includes the following:

Reagent A: 120 mM Tris, pH 7.5±0.3, containing 2.5 mM EDTA and 0.033% Tween-20

Reagent B: 14.5 mM 14:0 NPS-PC (1-myristoyl-2-(4-nitrophenylsuccinyl)-sn-glycero-3-phosphocholine) and 0.006-0.012 mM darapladib in DMSO

Volume: 4-17% of human serum or plasma, 79% of reagent A and 4.2% of reagent A.

Protocol: mix 20 μL of human serum/plasma or human albumin standard with 95 μL of reagent A. Add 5 μL of reagent B to start the reaction. Read kinetics of the reaction at 405 nm for 5-30 minutes or read end point after incubation at ambient temperature or slightly heated temperature such as 25-40° C. for 5-30 minutes.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. A method for determining the amount of albumin in a sample, the method comprising:
treating the sample with an esterase inhibitor that selectively inhibits non-albumin esterase activity;
combining the sample with a selective substrate of albumin, which has a carboxylic ester bond, so that the carboxylic ester bond is cleaved to generate a hydrolysate;
detecting the amount of the hydrolysate generated in a period of time; and
determining the amount of the albumin in the sample based on the amount of the hydrolysate in the period of time.

2. The method of claim 1, wherein the albumin is human serum albumin (HSA).

3. The method of claim 1, wherein the sample is blood, plasma, serum or urine.

4. The method of claim 1, wherein the selective substrate is a nitrophenyl ester of fatty acid or lipid.

5. The method of claim 1, wherein the selective substrate is 1-myristoyl-2-(4-nitrophenylsuccinyl)-sn-glycero-3-phosphocholine (14:0 NPS PC).

6. The method of claim 1, wherein the hydrolysate has a chromophore which enables effective detection of the hydrolysate via a light detector.

7. The method of claim 1, wherein the hydrolysate is nitrophenol.

8. The method of claim 7, wherein the hydrolysate is detected via a light detector at the wavelength of about 405 nm.

9. The method of claim 1, wherein the esterase inhibitor is a compound containing a sulfony fluoride group.

10. The method of claim 1, wherein the esterase inhibitor is 4-(2-aminoethyl)benzenesulfonyl fluoride hydrochloride or benzyl sulfonyl fluoride.

11. The method of claim 1, wherein the esterase inhibitor and the selective substrate are contained in one solution.
